(19) United States
(12) Patent Application Publication
(10) Pub. No.: US 2002/0198435 A1
(43) Pub. Date: Dec. 26, 2002

(54) Magnetic therapy devices and methods

(76) Inventor: Sumathi Paturu, Pleasant Grove, AL (US)

Correspondence Address: DR. SUMATHI PATURU, M.D., 207 BROOKE-LYN TERRACE, PLEASANT GROVE, AL, 35127, US

(21) Appl. No.: 10/196,925

(22) Filed: Jul. 16, 2002

Related U.S. Application Data
(63) Continuation-in-part of application No. 09/834,436, filed on Apr. 12, 2001.
Application No. 09/834,436 is a continuation-in-part of application No. 09/866,330, filed on May 24, 2001.

(51) Int. Cl. A61N 1/00

(52) U.S. Cl. 600/15

(57) ABSTRACT

The devices described in this invention relate generally to the use of magnets in the treatment of human diseases with an emphasis on the scientific basis of their modality of action.

The study of magnetic therapy to treat human diseases is an age old and on going discussion. So, many theories, both ancient and modern, were publicized in the past centuries, but none to the satisfaction of the scrutiny of the modern day medicine. The theories of the magnetic therapy so far popularized in the past decades were mostly based on Neuronal theories and also based on the different and unique properties of the North and the south poles.

However the theory postulated and applied in this present invention is a hematological and vascular phenomena based on the fact that Iron in the Hemoglobin molecule, contained within the red blood corpuscles, is attracted to the magnetic field. This results in the increased blood supply to the area under the effect of the magnetic field with the associated benefits of improved blood flow to the Penile circulation (in the case of erectile dysfunction) and improved Oxygenation in peripheral vascular insufficiency, as in the Pedal and cerebral circulations.

The modality of the electromagnetic therapy, and not the static magnetic therapy is employed in these devices. The principle of the Solenoids is being utilized in all the three embodiments being described, with Iron chosen as the material used for magnetization. The magnetization of the devices is synchronized with the patient's pulse (in 1:1 or 1:2 ratio), so that the freshly Oxygenated RBC gets to the magnetized area with each heart beat. Iron is known for its greater retention and so is easily magnetized, and it has low coercibility (resistance to demagnetization), and thus loses its magnetization easily, that makes it uniquely suitable as the metal chosen for the Solenoid in the embodiments described—the magnetic boot, the magnetic head piece and the magnetic sheath.

### BACKGROUND

This is a follow up application (because of the generically different species were filed together before) of the U.S. patent application filed on Apr. 12, 2001 (application Ser. No. 09/834436) and also it's continuation-in-part filed on May 24, 2001 with application Ser. No. 09/866330, both being the priority dates. The devices in this invention relate generally to the use of magnets in the treatment of human diseases with an emphasis on the scientific basis of their modality of action.

The study of magnetic therapy to treat human disease can be traced back as far as the early 16th century. Over the years, magnetic therapy has been alleged as a cure for diverse diseases and ailments ranging from cancer to chronic pain. The popularity of magnetic therapy continues today. However, despite the prevalence and popularity of magnetic therapy treatments, the physiological effects of magnetic therapy is still unsettled.

Magnetic fields have been historically described in relation to electric current. This relationship to electric current forms the basis of understanding the properties of magnets. All atoms are composed of protons and neutrons, which reside in the nucleus of the atom, and electrons which move rapidly about the nucleus of the atom. As the electrons are negatively charged, each electron generates its own magnetic moment, or magnetic dipole. These magnetic dipoles can be oriented in either of two opposing directions. However, not all atoms demonstrate magnetic properties. This is because many atoms have electrons that are paired with electrons of opposite magnetic dipoles, the net effect being the cancellation of the magnetic dipoles. These atoms are referred to as diamagnetic. Other atoms have unpaired electrons and possess a net magnetic dipole. These atoms do exhibit magnetic properties and are referred to as paramagnetic. Iron is an example of a paramagnetic atom. However, in some cases, the individual magnetic dipoles behave cooperatively and align themselves in the same direction to form magnetic domains. The compounds composed of these atoms demonstrate strong magnetic properties and are referred to as ferromagnetic. Ferromagnetic compounds include iron, cobalt, nickel, samarium, dysprosium and gadolinium.

Magnets always exist as dipoles, with a north pole and a south pole. Magnetic filed lines emerge from the north pole and converge at the south pole. The force of a magnetic field line is known as the magnetic flux and is measured in weber (w). The strength of a magnetic field, or magnetic flux density, is the number of magnetic field lines passing through a unit area and is measured in Telsa (T), or gauss (g).

There are two types of magnetic therapy: electromagnetic therapy and static magnetic field therapy. The types of magnetic fields generated in each of these types of therapy can be different. For example, electromagnetic therapy can employ a pulsating magnet field which allows the strength of the magnetic field to be regulated by controlling the flow of current, while in a static magnetic field the strength of the magnetic field does not vary. Electromagnetic therapy is based on the principle discovered by Michael Faraday that described the relationship between the movement of a magnetic and an electric field (electromagnetic induction). Faraday observed that passing a magnet in and out of a conducting electric coil produced voltage.

It has been known for some time that electrical activity in some form is involved in many aspects of human physiology. For instance, electrical activity has been measured during the regeneration of bone. In addition, it is well documented that many cellular responses are dictated by electrical gradients generated in the cell (for example, nerve cells). Therefore, it is possible that exposure of the human body to an pulsating electromagnetic field could produce a beneficial physiological response in the body. In fact, several studies have shown beneficial effects of pulsating electromagnetic field therapy in stimulating osteogenesis. The United States Food and Drug Administration has recently approved the use of pulsating electromagnetic field therapy for the treatment of some types of bone fractures

Various mechanism have been proposed for the effects of static magnetic field therapy, but none have achieved widespread acceptance. However, whatever the mechanism, the beneficial effects of the static magnetic field therapy could most probably be the result of increased blood flow to the area of the body treated with the static magnetic field.

It is well established that magnets can attract various types of metals, including iron. In the body, iron is prevalent in many places, including the blood. Blood cells contain hemoglobin molecules. Hemoglobin molecules function to transport oxygen from the lungs to the tissues of the body. Hemoglobin is composed of four subunits, with each subunit containing one molecule of iron, for a total of four iron molecules per hemoglobin molecule. Iron is paramagnetic. As a result, iron possesses a weak magnetization in the direction of an induced magnetic field. In addition, there are other paramagnetic materials present in the blood, including oxygen, sodium and potassium.

The body of a 70 kg man contains approximately 4 grams of iron, with 65%, or about 2.6 grams, being present in the hemoglobin. Therefore, hemoglobin molecules in the blood may contain enough iron to make the red blood cells of the blood responsive to magnetic fields and move, or be pulled, in the direction of an applied magnetic field.

Without being limited to other possible theories, the disclosure contemplates that the therapeutic benefits of static magnetic therapy and electromagnetic therapy that have been observed are mainly the result of increasing the blood circulation in the areas affected by magnetic induction through the attraction of the iron molecules in the hemoglobin molecules. This increased blood circulation may be the result of the attraction of the hemoglobin in the oxygen bound state or the oxygen free state.

In the two following embodiments being described, static magnetic therapy is the modality that is being applied in treating the vascular pathology. Examples of such include those associated with coronary or other vascular areas due to atherosclerosis, thrombosis, or mechanical trauma that injure vascular intima and set up thrombotic reaction.

Virchow's Triad says that the thrombus formation depends on viscosity of the blood, injury to the vessel wall and the velocity of blood flow. Here, we're trying to increase the velocity of blood flow in the injured or affected area, trying to eliminate at least one of the contributing factors of the Triad.

### 1) Vasvular Magnetic Cuff

Description

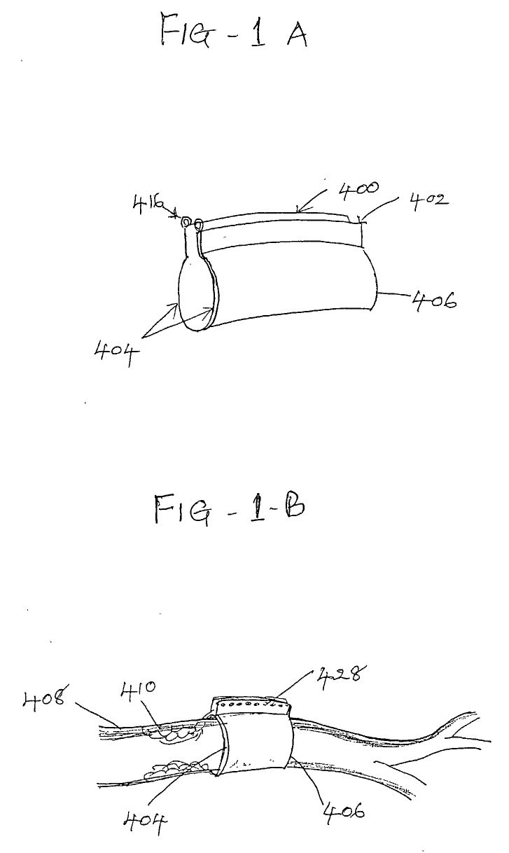

See FIGS. 1A & B. It is for external use (i.e. external to the vessel) to be encircled around the artery just distal to the area of pathology or area of anastamosis following surgery like CABG (Coronary Artery Bypass grafting) and other vascular anastamosis where postoperative thrombosis, narrowing or occlusion is usually a threatened complication. These cuffs can be applied distal to diseased arteries of smaller caliber that are not amenable to surgery during the time bigger arterial pathology is tackled surgically by Thorocotormy. Or it can be done endoscopically (by Mediastinoscope) when early lesions are diagnosed by cardiac catheterization to prevent further progression of the blockage that would need CABG at a later date which is associated with increased mortality and morbidity. It is like substituting Laparoscopy for Laparatomy with a benefit of preventing or delaying the progression of the block that necessitates CABG. This is especially beneficial for patients who are at high risk for very invasive prolonged surgery like CABG.

The magnet in the preferred embodiment is a flexible bio-compatible material such as rubber, impregnated with magnetic particles. The device has a first end 400 and second end 402, inner and outer side (not labeled), and also, proximal 404 and distal end 406. The magnet is wrapped around the artery 408 at the desired location and secured in place by joining the first and second ends of the magnet together to create a seal 428. In this manner, the magnet forms a cylindrical structure around the artery. The first and second ends can be joined together by convenient means such as a snapping device or stapling. To prevent injury to the vessel wall during approximation, this site is located conveniently farther from the vessel and the rest of the circumference of the magnetic cuff. The strength of the magnet is such that the magnetic field produced is effective in drawing the circulation from the area of pathology 410. The magnetic device can be of various lengths depending upon the strength of the magnetic field desired and other factors. The magnetic device is so constructed that there is a gradient of magnetic strength along the magnetic cuff with maximum strength in the proximal end 404, which gradually tapers to be minimum at the distal end 406. Therefore, the magnetic fields produced by this embodiment is greatest in the proximal end 404 adjacent to the area affected, and declines gradually along the length of the cuff to a minimum at the distal end (weaning of the magnetic field). Such a gradient facilitates the unimpeded flow of blood, directing it distal to the device so that in the area of the device itself, the blood flow would not be stagnant due to attraction exerted by the magnetic field. So, the ultimate effect is to enhance the velocity of blood flow in the area of the pathology and at the same time, not to stagnate it in the area of the magnetic device distal to it. The proximal side of the first and second ends have small rings 416 to anchor to adjacent solid structures to prevent sliding distally.

In any situation, if at all the magnetic cuff has to be used around a vein, it has to be used proximal to the area of the pathology, unlike in the arteries where it is used distally.

The magnetic field strength chosen in the cuff is important. As the proximal area of the cuff 404 is the area of maximum strength, it has to be so chosen it's magnetic field strength should be exerted to as far as the proximal end of the lesion 410 and a fraction proximal to it i.e. the magnetic field strength should cover the whole area of the pathology. In other words the velocity of the blood should start increasing before it approaches the area of the lesion (Rapid Bypass).

Patients should be informed to tell their doctors about the device if a MRI is advised to them in the future.

### 2) The Magnetic Stent Appendage

Description

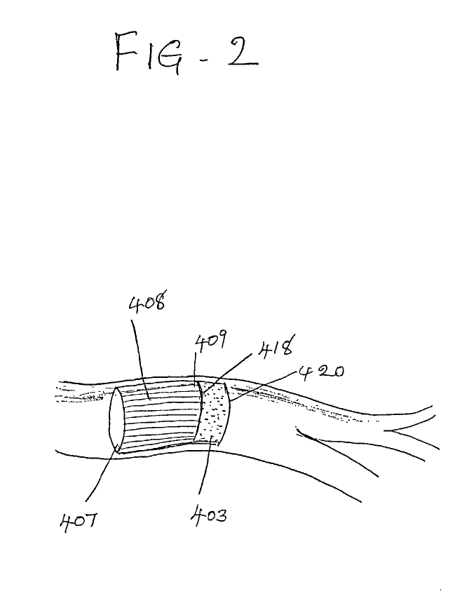

See the FIG. 2 of the magnetic appendage 403 attached to the distal end 409 of the Coronary Stent 408, which is a schematic but not the actual representation of the unit, because there can be so many different configurations of the stents.

As mentioned static magnetic therapy is the modality used in the magnetic appendage and also one of the principles of Virchow's triad is being employed here i.e. by increasing the velocity of the blood in the effected area we are trying to minimize the inflammatory response and inhibition of excessive fibrocellular neointimal formation.

The sent appendage is a biocompatable internal magnetic device used internally within the artery during the balloon angioplasty and stent placement. Most of the conventional stents are made of stainless steel and there are different configurations available. Neodymium, a rare metal has a higher saturation level and can be a stronger permanent magnet but it's biocompatability within the body needs to be tested. However the magnetic field strength produced by steel should be sufficient. The appendage can be ¼-⅓ of the length of the stent and can be configured similarly and shaped like a cylindrical appendage 403 attached to the stent by extremely thin attachments. The stent has a proximal end 407 and a distal end 409 and the distal end is attached to the proximal end 418 of the magnetic appendage. However it can be understood, during placement the distal end of the appendage (i.e. to be located distally in the artery after placement) forms the leading part of the stent during the procedure.

As in the magnetic cuff, the magnetic appendage also has a progressive gradient in the magnetic strength, being maximum at it's proximal end 418 and gradually declines towards the distal end 420 (weaning of the magnetic field). It can be done in different ways. The appendage can be made of segmental units of different magnetic field strengths put together to form the appendage, the lower field strengths being placed distally. The distal segments can be made gradually smaller (it also suits the caliber of the vasculature) which helps in decreasing the magnetic force.

The magnetic field strength chosen is important. As the proximal end of the appendage 418 is the area of the maximum strength, it has to be so chosen, it's maximum field strength should be exerted to as far as the proximal end 407 of the stent and a fraction beyond. In other words the magnetic field strength of the proximal end of the appendage should cover the whole of the stent and a fraction of an area beyond it (i.e. beyond the proximal area of the injured vessel wall).

As already mentioned such falling gradient of magnetic field facilitates—

1) Increased velocity of the blood starting in the proximal area of the stent and also all through it's length i.e. all through the surgically traumatized area (Rapid Bypass).

2) Unimpeded flow of blood in the area of the appendage itself over coming the magnetic field—due to falling gradient of the magnetic field which would also be over come by the blood pressure exerted with in the coronary arteries.

So the ultimate effect as stated before is to enhance the velocity of the blood flow in the area of the stent and at the same time not causing stagnation of the blood in the area of the appendage distal to it.

Patients should be informed to tell their doctor about the device if they are advised MRI in the future.

1. A device to create a magnetic field around a vein or artery, the device comprising a strip of magnetic material, and comprising of first and second ends, inner and outer surfaces, and also proximal and distal ends.

2. The device of claim 1 where the strip of magnetic material is bio-compatible.

3. The device of claim 1 where the strength of the magnetic field around the vein or artery is altered by varying the length of the strip of magnetic material.

4. The device of claim 1 further comprising a means for securing, the first end of the strip to the second end of the strip so that a generally cylindrical structure is formed around the vein or artery.

5. The device of claim 4 where the means for securing is a snapping device or stapling.

6. The device of claim 1 where the magnetic material has a highest magnetic strength in the proximal end (to cover the whole of the lesion) and tapers to a minimum distally.

7. The device & method of claim 1 and 5 where the proximal side of the first and the second ends have small rings 416 or holes within the substance to anchor to adjacent solid structures (to the myocardial tissue) to prevent sliding distally

8. The method of claim 7 where the device is applied by an endoscopic procedure or during a surgical procedure directed to treating severe blocks as in bypass surgery.

9. A magnetic device to be attached as an extension to the stent used during coronary balloon angioplasty and stent placement.

10. The device of claim 9 made of biocompatable permanent magnet, the structural configuration of which can be similar to the stent.

11. The device of claim 9 having varying magnetic gradient strength being maximum in the proximal end (to cover the whole of the stent) and tapers to a minimum distally.

12. The appendage of claim 9 can be ¼ to ⅓ of the length of the stent itself.
